(19) United States
(12) Patent Application Publication
(10) Pub. No.: US 2005/0048119 A1
(43) Pub. Date: Mar. 3, 2005

(54) Controlled release composition with semi-permeable membrane and poloxamer flux enhancer

(76) Inventors: Avinash Nangia, Lincoln, RI (US); Boyong Li, Morgantown, WV (US); Aaron Dely, Lighthouse Point, FL (US); Unchalee Lodin, North Miami Beach, FL (US); John R. Cardinal, Tamarac, FL (US)

Correspondence Address: HEDMAN & COSTIGAN P.C., 1185 AVENUE OF THE AMERICAS, NEW YORK, NY, 10036, US

(21) Appl. No.: 10/940,588

(22) Filed: Sep. 14, 2004

Related U.S. Application Data
(63) Continuation-in-part of application No. 10/664,803, filed on Sep. 19, 2003.
(60) Provisional application No. 60/412,180, filed on Sep. 20, 2002.
(60) Provisional application No. 60/412,181, filed on Sep. 20, 2002.

(51) Int. Cl. A61K 9/20; A61K 9/24; A61K 9/14

(52) U.S. Cl. 424/473

(57) ABSTRACT

A controlled release pharmaceutical tablet comprising a core containing the antihyperglycemic drug, a water soluble seal coat, a semipermeable membrane coating the core and at least one passageway in the membrane.

### CROSS REFERENCE TO RELATED APPLICATION

This is a continuation-in-part of U.S. patent application Ser. No. 10/664,803 filed on Sep. 19, 2003 and claims the benefit of provisional patent applications Ser. No. 60/412,180 and 60/412,181 filed on Sep. 20, 2002.

### BACKGROUND OF THE INVENTION

The present invention relates to a controlled release unit dose formulation containing a semipermeable membrane comprising a poloxamer flux enhancer. More specifically, the present invention relates to an oral dosage form comprising a water soluble drug, preferably an antihyperglycemic drug such as metformin or buformin, or a pharmaceutically acceptable salt thereof such as metformin hydrochloride or the metformin salts described in U.S. Pat. Nos. 3,957,853 and 4,080,472, which are incorporated herein by reference.

Many techniques have been used in the prior art to provide controlled and extended-release pharmaceutical dosage forms in order to achieve the dual goal of maintaining therapeutic serum levels of medicaments and maximizing patient compliance.

The prior art teaches extended release tablets that have an osmotically active drug core surrounded by a semipermeable membrane. These tablets function by allowing a fluid such as gastric or intestinal fluid to permeate the coating membrane and dissolve the active ingredient, thereby allowing the active ingredient to be released through a passageway in the coating membrane. Alternatively, if the active ingredient is insoluble in the permeating fluid, an expanding agent such as a hydrogel may push it through the passageway. Some representative examples of these osmotic tablet systems can be found in U.S. Pat. Nos. 3,845,770, 3,916,899, 4,034,758, 4,077,407 and 4,783,337. U.S. Pat. No. 3,952,741 teaches an osmotic device wherein the active agent is released from a core surrounded by a semipermeable membrane only after sufficient pressure has developed within the membrane to burst or rupture the membrane at a weak portion of the membrane.

The basic osmotic device described in the above cited patents have been refined over time in an effort to provide greater control of the release of the active ingredient. For example U.S. Pat. Nos. 4,777,049 and 4,851,229 describe an osmotic dosage form comprising a semipermeable wall surrounding a core. The core contains an active ingredient and a modulating agent wherein the modulating agent causes the active ingredient to be released through a passageway in the semipermeable membrane in a pulsed manner. Further refinements have included modifications to the semipermeable membrane surrounding the active core such as varying the proportions of the components that form the membrane, i.e. U.S. Pat. Nos. 5,178,867, 4,587,117 and 4,522,625 or increasing the number of coatings surrounding the active core, i.e., U.S. Pat. Nos. 5,650,170 and 4,892,739.

U.S. Pat. Nos. 6,099,859; 6,284,275; 6,495,162 and U.S. patent application Ser. No. 09/594,637 taught a controlled or sustained release formulation for an antihyperglycemic drug wherein the bioavailability of the drug is not decreased by the presence of food, the dosage form does not employ an expanding polymer, it can provide continuous and non-pulsating therapeutic levels of an antihyperglycemic drug to an animal or human in need of such treatment over a twelve hour to twenty-four hour period, and it provides a controlled or sustained release formulation for an antihyperglycemic drug that obtains peak plasma levels approximately 8-12 hours after administration. Furthermore, the osmotic core component, as taught by the above references, may be made using ordinary tablet compression techniques.

It is an object of this invention to provide a controlled release pharmaceutical tablet with a semipermeable membrane that employs a poloxamer to act as a flux enhancer.

It is also an object of this invention to provide controlled release pharmaceutical tablet wherein the plasticizer and the flux enhancer are the same excipient, namely, poloxamer.

It is a further object of this invention to provide a controlled release pharmaceutical tablet that uses a simpler membrane composition and produces a more reproducible drug release.

### SUMMARY OF THE INVENTION

The foregoing objectives are met by a controlled release dosage form comprising:
(a) a core comprising:
   (i) a drug, preferably a water soluble drug and most preferably an antihyperglycemic drug;
   (ii) at least one pharmaceutical excipient;
(b) optionally a water soluble seal coat;
(c) a semipermeable membrane coating surrounding the core comprising a poloxamer; and
(d) at least one passageway in the semipermeable membrane.

The dosage form of the present invention can provide therapeutic levels of the drug for twelve to twenty-four hour periods. It is believed that the poloxamer creates a semipermeable membrane with improved mechanical strength and elasticity. It is also believed that when the poloxamer serves as both the flux enhancer and the plasticizer the semipermeable membrane provides more consistent drug release as opposed to a semipermeable membrane coated dosage form containing separate flux enhancers and plasticizers.

### BRIEF DESCRIPTION OF THE DRAWINGS

FIG. 1. Is a cross section diagram of a metformin controlled release tablet of the invention comprising an active core, a seal coat, a semipermeable coat, two orifices, and a color coat.

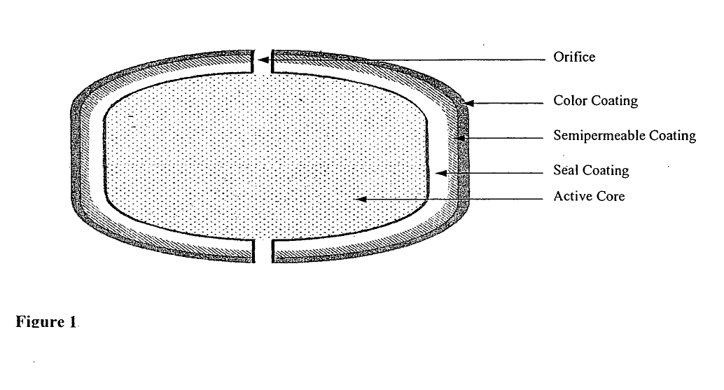

FIG. 2. Is a dissolution profile of metformin HCl controlled release tablets prepared in accordance with the present invention and coated with a semipermeable membrane employing different percentages of poloxamer (using a USP Type 1 apparatus at 100 rpm with a sinker in a phosphate buffer with a pH of 7.5).

FIG. 3. Is a dissolution profile of metformin HCl controlled release tablets prepared in accordance with the present invention and coated with a semipermeable membrane with 20% poloxamer (using a USP Type 1 apparatus at 100 rpm with a sinker in a phosphate buffer with a pH of 7.5).

### DETAILED DESCRIPTION OF THE INVENTION

The drug or active pharmaceutical ingredient can be any drug such as those described in Remington: The Science and Practice of Pharmacy (20^{th} Ed. 2000) or the U.S. Pharmacopoeia (26^{th} Ed. 2002), which are incorporated herein by reference. In a preferred embodiment the drug should be water soluble.

Drugs that are soluble in water and can be delivered by this invention include prochlorperazine edisylate, ferrous sulfate, amphetamine sulfate, benzphetamine hydrochloride, isoproteronol sulfate, aminocaproic acid, potassium chloride, mecaxylamine hydrochloride, procainamide hydrochloride, methamphetamine hydrochloride, phenmetrazine hydrochloride, bethanechol chloride, methacholine chloride, tridihexethyl chloride, phenformin hydrochloride, methylphenidate hydrochloride, pilocarpine hydrochloride, atropine sulfate, scopolamine bromide, isopropamide iodide, cimetidine hydrochloride, theophylline cholinate, cephalexin hydrochloride, and the like.

The drug can be in various forms, such as uncharged molecules, molecular complexes, pharmacologically acceptable salts such as hydrochloride, hydrobromide, sulfate, laurate, palmitate, tartrate, oleate, phosphate, nitrite, borate, acetate, maleate and salicylate. For acidic drugs, salts of metals, amines or organic cations; for example, quartemary ammonium can be used. Derivatives of drugs such as ester, ethers and amides can also be used. Additionally, a drug that is water insoluble can be used in a form that is a water soluble derivative thereof to serve as a solute, and on its release from tablet, is converted by enzymes, hydrolyzed by body pH or other metabolic processes to the original biologically active form.

Examples of other drugs that can be delivered by this invention include aspirin, indomethacin, naproxen, imipramine, levodopa, chloropromazine, methyldopa, dihydroxyphenylalanine, nitroglycerin, isosorbide dinitrate, propranolol, timolol, atenolol, alprenolol, cimetidine, fenoprofen, sulindac, indoprofen, clonidine, pivaloyloxyeihyl ester of alpha-methyldopa hydrochloride, theophylline, mefenamic, flufenamic, difuninal, nimodipine, nitrendipine, nisoldipine, nicardipine, felodipine, lidoflazine, tiapamil, gallopamil, amlodipine, mioflazine, calcium gluconate, ketoprofen, ibuprofen, cephalexin, erythromycin, quanbenz, hydrochlorothiazide, ranitidine, flurbiprofen, fenbufen, fluprofen, tolmetin, haloperidol, zomepirac, chlordiazepoxide hydrochloride, diazepan, amitriptylin hydrochloride, impramine hydrochloride, imipramine pamoate, captopril, ramipril, endlapriat, famotidine, nizatidine, sucralfate, ferrous lactate, vincamine, diazepam, phenoxybenzamine, diltiazem, milrinone, captopril, madol, alolofenac, lisinopril, enalapril, etintidine, tertatolol, minoxidil, chlordiazepoxide, and the like.

Examples of other relatively soluble drugs which may be included in the controlled release formulations of the present invention include vasodilators (e.g., papaverine, diltiazem), cholinergics (e.g., neostigmine, pyridostigmine), antihistamines (e.g., dimenhydrinate, diphenhydramine, chlorpheniramine and dexchlorpheniramine maleate), non-steroidal anti-inflammatory agents (e.g., naproxen, diclofenac, ibuprofen, aspirin, sulindac), gastrointestinals and anti-emetics (e.g., metoclopramide), analgesics (e.g., aspirin, codeine, morphine, dihydromorphone, oxycodone, etc.), anti-epileptics (e.g., phenyloin, meprobamate and nitrezepam), anti-tussive agents and expectorants (e.g., codeine phosphate), antituberculosis agents (e.g., isoniazid), anti-spasmodics (e.g. atropine, scopolamine), diuretics (e.g., bendrofluazide), anti-hypertensives (e.g., propranolol, clonidine), bronchodilators (e.g., albuterol), laxatives, antacids, vitamins (e.g., ascorbic acid), sympathomimetics (e.g., ephedrine, phenylpropanolamine), iron preparations (e.g., ferrous gluconate), anti-muscarinics (e.g., anisotropine), hormones (e.g., insulin, heparin), anti-inflammatory steroids (e.g., hydrocortisone, triamcinolone, prednisone), antibiotics (e.g., penicillin v, tetracycline, clindamycin, novobiocin, metronidazde, cloxacillin), antihemorrhoidals, antidiarrheals, mucolytics, sedatives and decongestants. The above list is not exhaustive.

In an alternative embodiment of the present invention the drug employed in the core is an antihyperglycemic drug. The term antihyperglycemic drugs as used in this specification refers to drugs that are useful in controlling or managing noninsulin-dependent diabetes mellitus (NIDDM). Preferably, the antihyperglycemic drug is a biguanide such as metformin or buformin or a pharmaceutically acceptable salt thereof such as metformin hydrochloride.

In addition to the drug, the core should contain at least one pharmaceutical excipient such as a binder, absorption enhancer, diluent, flow aid, lubricant, osmopolymer, osmagent and combinations of the foregoing.

The binding agent may be any conventionally known pharmaceutically acceptable binder such as polyvinyl pyrrolidone, hydroxypropyl cellulose, hydroxyethyl cellulose, ethylcellulose, polymethacrylate, waxes and the like. Mixtures of the aforementioned binding agents may also be used. The preferred binding agents are water soluble such as polyvinyl pyrrolidone having an average molecular weight of 25,000 to 3,000,000. Polyvinyl pyrrolidone is commercially available as POVIDONE®. If a binding agent is used it should comprise approximately about 0 to about 40% of the total weight of the core and preferably about 3% to about 15% of the total weight of the core.

The core may optionally comprise an absorption enhancer. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(β-aminoethyl ether)-N,N,N,N-tetraacetic acid (EGTA). The preferred absorption enhancer is soldium lauryl sulfate. If an absorption enhance is used in the core it should comprise approximately 0 to about 20% of the total weight of the core and most preferably about 2% to about 10% of the total weight of the core.

The core may also contain a diluent or filler. The diluent may be any conventionally known pharmaceutically acceptable diluent such lactose, dextrose, sucrose maltose, fructose, galactose, microcrystalline cellulose, gelatin, polyvinylpyrrolidone, rice starch, corn starch, calcium carbonate and the like or mixtures thereof. If a diluent is used in the core it should comprise approximately 0% to about 40% of the total weight of the core, preferably about 1% to about 30% of the total weight of the core and most preferably about 2% to about 20% of the total weight of the core.

Suitable lubricants which can be used in preparing compressed forms of the present invention may include talc, stearic acid, magnesium stearate, glyceryl monostearate, crospovidone, sodium stearyl fumerate, hydrogenated oils, polyethylene glycols and sodium stearate.

Suitable flow aids which can also be used in the present invention may include talc, silica, and metallic stearates

The core may also contain an osmopolymer. Osmopolymers interact with water and aqueous biological fluids and swell or expand to an equilibrium state. Osmopolymers exhibit the ability to swell in water and to retain a significant portion of the imbibed and absorbed water within a polymer structure. Suitable osmopolymer include, but are not limited to, hydroxypropyl methylcellulose, alkylcellulose, hydroxyalkylcellulose, poly(alkylene oxide), or combinations thereof. Other examples of osmopolymers are provided in U.S. Pat. Nos. 4,612,008; 4,327,725; and 5,082,668; which are incorporated herein by reference. An osmopolymer can also function as a binding agent for the core.

The core may also contain an osmagent. An osmagent is a material which attracts fluid into the core of a pharmaceutical tablet. Materials which may be suitable as osmagents include electrolytes and organic acids. Example of useful materials include simple sugars, such as lactose and sucrose, salts such as magnesium sulfate, potassium chloride, ammonium chloride, calcium sulfate, sodium chloride, calcium lactate, mannitol, urea, inositol, magnesium succinate, lithium chloride, lithium sulfate, potassium sulfate, sodium carbonate, sodium sulfate, potassium acid phosphate, tartaric acid, citric acid, itaconic acid, fumaric acid, lactic acid, ascorbic acid, malic acid, maleic acid and the like or combinations thereof. Other osmagents are described in U.S. Pat. Nos. 4,612,008 and 5,082,668; which are incorporated herein by reference.

In a preferred embodiment of the present invention, the core comprises an antihyperglycemic drug, a binder and an absorption enhancer. The core is preferably formed by wet granulating the core ingredients and compressing the granules with the addition of a lubricant into a tablet on a rotary press. The core may also be formed by dry granulating the core ingredients and compressing the granules with the addition of a lubricant into tablets. It may also be formed by direct compression.

The homogeneous core is coated with a seal coat, preferably a water soluble seal coat, such as OPADRY® Clear. The seal coat is used to protect the core during the remainder of the tablet processing. OPADRY® is a coating system which combines polymers, plasticizers and, if desired, pigments. The seal coat may also comprise an osmotic agent, such as the osmagents described in U.S. Pat. No. 5,916,596, which is incorporated herein by reference.

The seal coated core is coated with a semipermeable membrane, preferably a modified polymeric membrane to form the controlled release tablet of the invention. The semipermeable membrane is permeable to the passage of an external fluid such as water and biological fluids. Additionally, the membrane is impermeable to the passage of the antihyperglycemic drug in the core. Water insoluble polymers that are useful in forming the semipermeable membrane are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,612,008, which are incorporated herein by reference. The most preferred water insoluble polymer is cellulose acetate comprising an acetyl content of 39.3% to 40.3%. This product is commercially available from Eastman Fine Chemicals.

The semipermeable membrane can be formed by the above-described polymers in combination with a flux enhancing agent. The flux enhancing agent increases the volume of fluid imbibed into the core to enable the dosage form to dispense substantially all of the antihyperglycemic drug through the passageway and/or the porous membrane. The flux enhancing agent can be a water soluble material or an enteric material. Some examples of the preferred materials that are useful as flux enhancers are sodium chloride, potassium chloride, sucrose, sorbitol, mannitol, polyethylene glycol (PEG), poloxamer (LUTROL®) propylene glycol, hydroxypropyl cellulose, hydroxypropyl methycellulose, hydroxypropyl methycellulose phthalate, cellulose acetate phthalate, polyvinyl alcohols, methacrylic acid copolymers and mixtures thereof. The preferred flux enhancer is poloxamer such as those described in Gilbert, J. C. et al., Controlled Release From Erodible Pluronic Matrices, Proceed. Intern. Symp. Control. Rel. Bioact. Mater., vol. 18, p. 573-74 (1991); Arthur M. Kibbe, Handbook of Pharmaceutical Excipients, p. 386-88 (3^{rd} Ed. 1999) and Kabanov, Alexander V. et al., Pluronic® Block Copolymers As Novel Polymer Therapeutics For Drug And Gene Delivery, Journal of Controlled Release, vol. 82, p. 189-212 (2002); which are incorporated herein by reference.

The flux enhancing agent comprises approximately 0% to about 40% of the total weight of the coating, most preferably about 2% to about 20% of the total weight of the coating. The flux enhancing agent dissolves or leaches from the semipermeable membrane to form paths in the semipermeable membrane for the fluid to enter the core and dissolve the active ingredient. A controlled release dosage form should ideally deliver substantially all of the drug from the dosage form to the environment of use. However, a common problem encountered in osmotic dosage forms is that residual drug is left in the tablet interior which is not available for absorption. Often an increased amount of drug is added to compensate for this residual drug in the system.

The usage of flux enhancers provides an added benefit in that it helps ensure that all of the drug is released, as is shown in the present invention.

The semipermeable membrane may also be formed with other commonly known excipients such plasticizers. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are poloxamer, triacetin, acetylated monoglyceride, grape seed oil, olive oil, sesame oil, acetyltributylcitrate, acetyltriethylcitrate, glycerin sorbitol, diethyloxalate, diethylmalate, diethyl fumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating. The preferred plasticizer is poloxamer as described herein.

Alternatively, the present invention may employ an excipient that acts as both a flux enhancer and as a plasticizer. The excipient poloxamer, commercially available from BASF as LUTROL® may be used to achieve both the purposes of a plasticizer and a flux enhancer. Poloxamers are a group of structurally related block ABA copolymers, also known commercially as PLURONICS®. These products are non-toxic in oral dosage forms and highly efficient solublizers. Poloxamers are also known to form gels in aqueous solutions. When these excipients gel in aqueous solutions they form a barrier to diffusion, which then erodes as the polymer dissolves. Therefore, by altering the amount of the poloxamer and its molecular weight one is capable of influencing the release profile of a pharmaceutical dosage forms. See Gilbert, J. C. et al., Controlled Release From Erodible Pluronic Matrices, Proceed. Intern. Symp. Control. Rel; Bioact. Mater., vol. 18, p. 573-74 (1991); Arthur M. Kibbe, Handbook of Pharmaceutical Excipients, p. 386-88 (3^{rd} Ed. 1999) and Kabanov, Alexander V. et al., Pluronic® Block Copolymers As Novel Polymer Therapeutics For Drug And Gene Delivery, Journal of Controlled Release, vol. 82, p. 189-212 (2002).

As used herein the term passageway includes an aperture, orifice, bore, hole, weakened area or an erodible element such as a gelatin plug that erodes to form an osmotic passageway for the release of the antihyperglycemic drug from the dosage form. A detailed description of the Sustained Release Coating passageway can be found in U.S. Pat. Nos. such as U.S. Pat. Nos. 3,845,770, 3,916,899, 4,034,758, 4,077,407,4,783,337 and 5,071,607.

Generally, the membrane coating around the core will comprise from about 1% to about 15% and preferably about 2% to about 5% based on the total weight of the core and coating.

In an alternative embodiment, the dosage form of the present invention may also comprise an effective amount of the drug that is available for immediate release. The effective amount of drug for immediate release may be coated onto the semipermeable membrane of the dosage form or it may be incorporated into the semipermeable membrane.

In a preferred embodiment the dosage form will have the following composition:

| | Preferred | Most Preferred |
|---|---|---|
| CORE: | | |
| drug | 50-98% | 75-95% |
| binder | 0-40% | 3-15% |
| absorption | 0-20% | 2-10% |
| enhancer | | |
| lubricant | 0-10% | 0-5% |
| COATING: | | |
| semipermeable | 50-99% | 75-95% |
| polymer | | |
| poloxamer | 0-80% | 4-35% |

### DESCRIPTION OF THE PREFERRED EMBODIMENTS

### EXAMPLE I

A controlled release tablet of metformin HCl is prepared as follows:

A. Granulation

| Ingredients | % | kg |
|---|---|---|
| Metformin Hydrochloride, BP | 93.29 | 139.94 |
| Povidone K90, USP | 6.71 | 10.06 |
| Purified Water, USP | — | 191.19 |
| Total | 100.00 | 150.00 |

The metformin HCl is delumped by passing it through a Comil equipped with a #813 screen (approximately equivalent to a 20 mesh size) and granulated in a Glatt GPCG-60 fluid bed coater with a 32″ Wurster column by spraying Povidone K-90 solution in purified water (bottom spray) at a spraying rate of 500-1200 g/min, a product temperature of 38-43° C. and an atomization air pressure of 2.5-3 bars. The granules are discharged, and then sized through a Comil equipped with a #1143 screen (approximately equivalent to a 15 mesh size).

B. Blending and Compression

| Ingredients | % | kg |
|---|---|---|
| Metformin HCl Granules | 94.922 | 149.89 |
| Sodium Lauryl Sulfate, NF | 4.577 | 7.228 |
| Magnesium Stearate, NF | 0.501 | 0.790 |
| Total | 100.00 | 157.91 |

Metformin HCl granules are blended with sodium lauryl sulfate in a 20-ft³ slant-cone blender and then blended with magnesium stearate. The blend is then compressed into tablets with tablet weights around 1129 mg on a 32-station rotary tablet press equipped with round standard concave ½″ tooling.

C. Seal Coating

| Ingredients | % | kg |
|---|---|---|
| Metformin HCl XT Tablet, | 96.00 | 52.91 |
| 1000 mg (uncoated) | | |
| Opadry Clear | 4.00 | 2.202 |
| Purified Water, USP | * | 19.813 |
| Total | 100.00 | 55.112 |

*Evaporated during processing.

The uncoated tablets are then seal coated in a 36″ coating pan with an Opadry Clear solution in purified water at 40-47° C. exhaust temperature, 50 psi atomization air pressure and 111-147 g/min spray rate.

D. Semipermeable Film Coating

| Ingredients | % | kg |
|---|---|---|
| Metformin HCL Tablets, 1000 mg (Seal Coated) | 97.50 | 58.50 |
| Cellulose Acetate 398-10, NF | 2.00 | 1.20 |
| Poloxamer, NF (Lutrol F-68) | 0.50 | 0.30 |
| Purified Water, USP | * | 1.46 |
| Acetone, NF | * | 47.05 |
| Total | 100.00 | 60.00 |

*Evaporated during processing.

The seal coated tablets are then coated in a Glatt GPCG-60 fluid-bed coater with an 18″ Wurster column with a cellulose acetate solution in acetone/water at a production temperature of 21-23° C., a spray rate of about 400 g/min and an atomization air pressure of about 2 bars.

E. Laser Drilling

The semipermeable film coated tablets are then drilled to form 0.5 mm orifices on each side of the tablets using a Duplex Laser Tablet Driller.

### EXAMPLE II

The seal coated metformin HCl tablets, 1000 mg can be made with a similar process as described in Example I with the following compositions:

Formulation of Metformin HCl Tablets, 1000 mg (Seal Coated)

| Ingredients | % |
|---|---|
| Core Tablet: | |
| Metformin Hydrochloride, BP | 85.01 |
| Sodium Lauryl Sulfate, NF | 4.39 |
| Povidone K90, USP | 6.12 |
| Magnesium Stearate, NF | 0.48 |
| Seal Coating: | |
| Opadry Clear | 4.00 |
| Total | 100.00 |

Semipermeable membrane coated metformin HCl Tablets, 1000 mg can be made with a similar process as described in Example I with the following formulations:

Formulation of Metformin HCl Tablet, 1000 mg, Coated with
Semipermeable Membrane with Different Percentages
of Poloxamer (LUTROL ®)

| Ingredients | 15% Lutrol | 20% Lutrol | 25% Lutrol |
|---|---|---|---|
| Metformin HCl Tablet, | 97.00 | 97.00 | 97.00 |
| 1000 mg (Seal Coated) | | | |
| Cellulose Acetate 398-10, NF | 2.55 | 2.40 | 2.25 |
| Poloxamer, NF (Lutrol F-68) | 0.45 | 0.60 | 0.75 |
| Total | 100.00 | 100.00 | 100.00 |

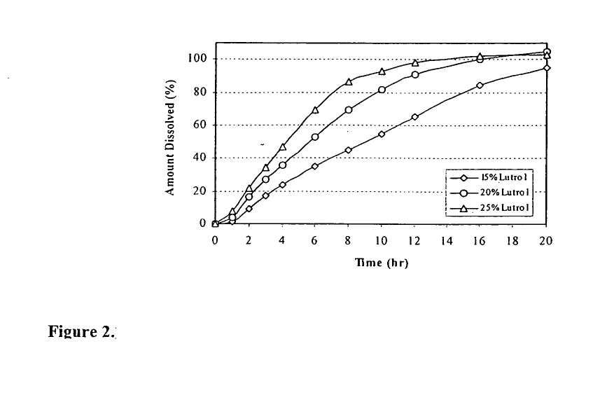

The dissolution profiles of metformin HCl controlled release tablets, 1000 mg coated with semipermeable membranes with different percentages of LUTROL® at 3% coating level (using a USP Type 1 apparatus at 100 rpm with a sinker in a pH 7.5 phosphate buffer) are given in FIG. 2.

### EXAMPLE III

The seal coated metformin HCl tablets, 1000 mg can be made with a similar process as described in Example I with the following compositions:

Formulation of Metformin HCl Tablets, 1000 mg (Seal Coated)

| Ingredients | Mg/tablet | % |
|---|---|---|
| Core Tablet: | | |
| Metformin Hydrochloride, BP | 1000 | 85.01 |
| Sodium Lauryl Sulfate, NF | 51.64 | 4.39 |
| Povidone K90, USP | 71.99 | 6.12 |
| Magnesium Stearate, NF | 5.646 | 0.48 |
| Seal Coating: | | |
| Opadry Clear | 47.05 | 4.00 |
| Total | 1176 | 100.00 |

Semipermeable membrane coated metformin HCl Tablets, 1000 mg can be made with a similar process as described in Example I with the following formulations:

Formulation of Metformin HCl Tablet, 1000 mg, Coated with
Semipermeable Membrane with 20% Poloxamer
(Lutrol) at Different Coating Levels (C.L.).

| Ingredients | 2% C.L. | 3% C.L. | 4% C.L. |
|---|---|---|---|
| Metformin HCl Tablet, 1000 mg | 98.00 | 97.00 | 96.00 |
| (Seal Coated) | | | |
| Cellulose Acetate 398-10, NF | 1.60 | 2.40 | 3.20 |
| Poloxamer, NF (Lutrol F-68) | 0.40 | 0.60 | 0.80 |
| Total | 100.00 | 100.00 | 100.00 |

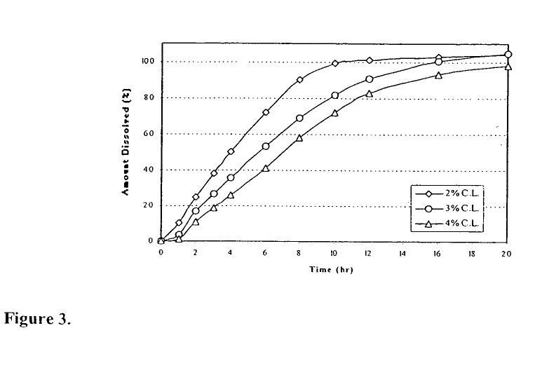

The dissolution profiles of Metformin HCl Controlled Release Tablets, 1000 mg coated with semipermeable membranes with 20% Lutrol at different coating levels (using a USP Type 1 apparatus at 100 rpm with a sinker in a pH 7.5 phosphate buffer) are given in FIG. 3.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A controlled release pharmaceutical tablet comprising:
(a) a core comprising:
(i) a drug;
(ii) at least one pharmaceutically acceptable excipient;
(b) optionally a water soluble seal coat;
(c) a semipermeable membrane coating covering said core wherein the membrane comprises:
i) a material that is permeable to the passage of water and biological fluids and is impermeable to the passage of the drug; and
(ii) a poloxamer.
(d) at least one passageway in the semipermeable membrane

2. A controlled release pharmaceutical tablet as defined in claim 1 wherein the drug is water soluble.

3. A controlled release pharmaceutical tablet as defined in claim 1 wherein the drug is an antihyperglycemic.

4. A controlled release pharmaceutical tablet as defined in claim 3 wherein the antihyperglycemic drug is a metformin or a pharmaceutically acceptable salt thereof.

5. A controlled release pharmaceutical tablet as defined in claim 3 wherein the antihyperglycemic drug is buformin or a pharmaceutically acceptable salt thereof.

6. A controlled release pharmaceutical tablet as defined in claim 1 wherein the pharmaceutical excipient in the core comprises a binding agent that is water soluble.

7. A controlled release pharmaceutical tablet as defined in claim 6 wherein the water soluble binding agent is polyvinyl pyrrolidone, hydroxypropyl cellulose, hydroxyethyl cellulose, waxes or mixtures thereof.

8. A controlled release pharmaceutical tablet as defined in claim 1 wherein the pharmaceutical excipient in the core comprises an absorption enhancer is selected from the group consisting of poloxamers, fatty acids, surfactants, chelating agents, bile salts or mixtures thereof.

9. A controlled release pharmaceutical tablet as defined in claim 1 wherein the material that is permeable to the passage of water an biological fluids is a water insoluble cellulose derivative.

10. A controlled release pharmaceutical tablet as defined in claim 13 wherein the water insoluble cellulose derivative in the membrane around the core is cellulose acetate.

11. A controlled release pharmaceutical tablet as defined in claim 1 wherein the semipermeable membrane further comprises a flux enhancer poloxamer.

12. A controlled release pharmaceutical tablet as defined in claim 15 wherein the second flux enhancer is sodium chloride, potassium chloride, sucrose, sorbitol, mannitol, polyethylene glycol, propylene glycol, hydroxypropyl cellulose, hydroxypropyl methycellulose, hydroxypropyl methycellulose phthalate, cellulose acetate phthalate, polyvinyl alcohols, methacrylic acid copolymers or mixtures thereof.

13. A controlled release pharmaceutical tablet as defined in claim 16 wherein the flux enhancer is poloxamer.

14. A controlled release pharmaceutical tablet as defined in claim 1 wherein the semipermeable membrane further comprises a second plasticizer in addition to the poloxamer.

15. A controlled release pharmaceutical tablet as defined in claim 1 wherein at least two passageways are formed in the semipermeable membrane.

16. A controlled release pharmaceutical tablet comprising:
(a) a core consisting essentially of:
(i) a drug;
(ii) a binding agent;
(iii) optionally an absorption enhancer; and
(iv) optionally a lubricant;
(b) optionally a water soluble seal coat;
(c) a semipermeable membrane coating covering said core consisting essentially of:
(i) a water insoluble cellulose derivative; and
(ii) a flux enhancer;
(d) at least one passageway in the semipermeable membrane.

17. A controlled release antihyperglycemic tablet comprising:
(a) a core consisting essentially of:
(i) metformin or a pharmaceutically acceptable salt thereof;
(ii) a water soluble binding agent; and
(iii) an absorption enhancer;
(b) optionally a water soluble seal coat;
(c) a semipermeable membrane coating covering said core comprising:
(i) cellulose acetate;
(ii) a poloxamer; and
(d) at least one passageway in the semipermeable membrane.

18. A controlled release pharmaceutical tablet consisting essentially of:
(a) a core comprising:
(i) 75-95% of an antihyperglycemic drug;
(ii) 3-15% of a binding agent; and
(iii) 2-15% of an absorption enhancer;
(b) a water soluble seal coat;
(c) a semipermeable membrane coating covering said core wherein the semipermeable membrane is permeable to the passage of water and biological fluids and is impermeable to the passage of the antihyperglycemic drug and comprises:
(i) 75-95% of a polymer;
(ii) 4-35% of a poloxamer; and
(d) at least one passageway in the semipermeable membrane for the release of the antihyperglycemic drug.
